SEQUENCE LISTING (11 sequences)

SEQ ID NO: 1 - protein, 19 aa, Artificial
  Synthetic
XXXCXXXCXXGXCXXXCXX

SEQ ID NO: 2 - protein, 18 aa, Artificial
  Synthetic
RRWCYRKCYKGYCYRKCR

SEQ ID NO: 3 - protein, 17 aa, Artificial
  Synthetic
KWAFRVAYRGIAYRRAR

SEQ ID NO: 4 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 5 - DNA, 3656 bp, Homo sapiens
agcgggagtcgcggaacagcaggcccgagcccaccgcgccgggccccggacgccgcgcggaaaagatgaatttacaaccaattttctggattggactgatcagttcagtttgctgtgtgtttgctcaaacagatgaaaatagatgtttaaaagcaaatgccaaatcatgtggagaatgtatacaagcagggccaaattgtgggtggtgcacaaattcaacatttttacaggaaggaatgcctacttctgcacgatgtgatgatttagaagccttaaaaaagaagggttgccctccagatgacatagaaaatcccagaggctccaaagatataaagaaaaataaaaatgtaaccaaccgtagcaaaggaacagcagagaagctcaagccagaggatattactcagatccaaccacagcagttggttttgcgattaagatcaggggagccacagacatttacattaaaattcaagagagctgaagactatcccattgacctctactaccttatggacctgtcttactcaatgaaagacgatttggagaatgtaaaaagtcttggaacagatctgatgaatgaaatgaggaggattacttcggacttcagaattggatttggctcatttgtggaaaagactgtgatgccttacattagcacaacaccagctaagctcaggaacccttgcacaagtgaacagaactgcaccagcccatttagctacaaaaatgtgctcagtcttactaataaaggagaagtatttaatgaacttgttggaaaacagcgcatatctggaaatttggattctccagaaggtggtttcgatgccatcatgcaagttgcagtttgtggatcactgattggctggaggaatgttacacggctgctggtgttttccacagatgccgggtttcactttgctggagatgggaaacttggtggcattgttttaccaaatgatggacaatgtcacctggaaaataatatgtacacaatgagccattattatgattatccttctattgctcaccttgtccagaaactgagtgaaaataatattcagacaatttttgcagttactgaagaatttcagcctgtttacaaggagctgaaaaacttgatccctaagtcagcagtaggaacattatctgcaaattctagcaatgtaattcagttgatcattgatgcatacaattccctttcctcagaagtcattttggaaaacggcaaattgtcagaaggagtaacaataagttacaaatcttactgcaagaacggggtgaatggaacaggggaaaatggaagaaaatgttccaatatttccattggagatgaggttcaatttgaaattagcataacttcaaataagtgtccaaaaaaggattctgacagctttaaaattaggcctctgggctttacggaggaagtagaggttattcttcagtacatctgtgaatgtgaatgccaaagcgaaggcatccctgaaagtcccaagtgtcatgaaggaaatgggacatttgagtgtggcgcgtgcaggtgcaatgaagggcgtgttggtagacattgtgaatgcagcacagatgaagttaacagtgaagacatggatgcttactgcaggaaagaaaacagttcagaaatctgcagtaacaatggagagtgcgtctgcggacagtgtgtttgtaggaagagggataatacaaatgaaatttattctggcaaattctgcgagtgtgataatttcaactgtgatagatccaatggcttaatttgtggaggaaatggtgtttgcaagtgtcgtgtgtgtgagtgcaaccccaactacactggcagtgcatgtgactgttctttggatactagtacttgtgaagccagcaacggacagatctgcaatggccggggcatctgtgagtgtggtgtctgtaagtgtacagatccgaagtttcaagggcaaacgtgtgagatgtgtcagacctgccttggtgtctgtgctgagcataaagaatgtgttcagtgcagagccttcaataaaggagaaaagaaagacacatgcacacaggaatgttcctattttaacattaccaaggtagaaagtcgggacaaattaccccagccggtccaacctgatcctgtgtcccattgtaaggagaaggatgttgacgactgttggttctattttacgtattcagtgaatgggaacaacgaggtcatggttcatgttgtggagaatccagagtgtcccactggtccagacatcattccaattgtagctggtgtggttgctggaattgttcttattggccttgcattactgctgatatggaagcttttaatgataattcatgacagaagggagtttgctaaatttgaaaaggagaaaatgaatgccaaatgggacacgggtgaaaatcctatttataagagtgccgtaacaactgtggtcaatccgaagtatgagggaaaatgagtactgcccgtgcaaatcccacaacactgaatgcaaagtagcaatttccatagtcacagttaggtagctttagggcaatattgccatggttttactcatgtgcaggttttgaaaatgtacaatatgtataatttttaaaatgttttattattttgaaaataatgttgtaattcatgccagggactgacaaaagacttgagacaggatggttattcttgtcagctaaggtcacattgtgcctttttgaccttttcttcctggactattgaaatcaagcttattggattaagtgatatttctatagcgattgaaagggcaatagttaaagtaatgagcatgatgagagtttctgttaatcatgtattaaaactgatttttagctttacaaatatgtcagtttgcagttatgcagaatccaaagtaaatgtcctgctagctagttaaggattgttttaaatctgttattttgctatttgcctgttagacatgactgatgacatatctgaaagacaagtatgttgagagttgctggtgtaaaatacgtttgaaatagttgatctacaaaggccatgggaaaaattcagagagttaggaaggaaaaaccaatagctttaaaacctgtgtgccattttaagagttacttaatgtttggtaacttttatgccttcactttacaaattcaagccttagataaaagaaccgagcaattttctgctaaaaagtccttgatttagcactatttacatacaggccatactttacaaagtatttgctgaatggggaccttttgagttgaatttattttattatttttattttgtttaatgtctggtgctttctatcacctcttctaatcttttaatgtatttgtttgcaattttggggtaagactttttttatgagtactttttctttgaagttttagcggtcaatttgcctttttaatgaacatgtgaagttatactgtggctatgcaacagctctcacctacgcgagtcttactttgagttagtgccataacagaccactgtatgtttacttctcaccatttgagttgcccatcttgtttcacactagtcacattcttgttttaagtgcctttagttttaacagttcactttttacagtgctatttactgaagttatttattaaatatgcctaaaatacttaaatcggaaaaaaaaaaaaaaaaa

SEQ ID NO: 6 - protein, 93 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 7 - DNA, 3524 bp, Homo sapiens
tctccgtcagccgcattgcccgctcggcgtccggcccccgacccgtgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatgtgagagggtcagacgcctgaggaacccttacagtaggagcccagctctgaaaccagtgttagggaagggcctgccacagcctcccctgccagggcagggccccaggcattgccaagggctttgttttgcacactttgccatattttcaccatttgattatgtagcaaaatacatgacatttatttttcatttagtttgattattcagtgtcactggcgacacgtagcagcttagactaaggccattattgtacttgccttattagagtgtctttccacggagccactcctctgactcagggctcctgggttttgtattctctgagctgtgcaggtggggagactgggctgagggagcctggccccatggtcagccctagggtggagagccaccaagagggacgcctgggggtgccaggaccagtcaacctgggcaaagcctagtgaaggcttctctctgtgggatgggatggtggagggccacatgggaggctcacccccttctccatccacatgggagccgggtctgcctcttctgggagggcagcagggctaccctgagctgaggcagcagtgtgaggccagggcagagtgagacccagccctcatcccgagcacctccacatcctccacgttctgctcatcattctctgtctcatccatcatcatgtgtgtccacgactgtctccatggccccgcaaaaggactctcaggaccaaagctttcatgtaaactgtgcaccaagcaggaaatgaaaatgtcttgtgttacctgaaaacactgtgcacatctgtgtcttgtgtggaatattgtccattgtccaatcctatgtttttgttcaaagccagcgtcctcctctgtgaccaatgtcttgatgcatgcactgttccccctgtgcagccgctgagcgaggagatgctccttgggccctttgagtgcagtcctgatcagagccgtggtcctttggggtgaactaccttggttcccccactgatcacaaaaacatggtgggtccatgggcagagcccaagggaattcggtgtgcaccagggttgaccccagaggattgctgccccatcagtgctccctcacatgtcagtaccttcaaactagggccaagcccagcactgcttgaggaaaacaagcattcacaacttgtttttggtttttaaaacccagtccacaaaataaccaatcctggacatgaagattctttcccaattcacatctaacctcatcttcttcaccatttggcaatgccatcatctcctgccttcctcctgggccctctctgctctgcgtgtcacctgtgcttcgggcccttcccacaggacatttctctaagagaacaatgtgctatgtgaagagtaagtcaacctgcctgacatttggagtgttcccctcccactgagggcagtcgatagagctgtattaagccacttaaaatgttcacttttgacaaaggcaagcacttgtgggtttttgttttgtttttcattcagtcttacgaatacttttgccctttgattaaagactccagttaaaaaaaattttaatgaagaaagtggaaaacaaggaagtcaaagcaaggaaactatgtaacatgtaggaagtaggaagtaaattatagtgatgtaatcttgaattgtaactgttcgtgaatttaataatctgtagggtaattagtaacatgtgttaagtattttcataagtatttcaaattggagcttcatggcagaaggcaaacccatcaacaaaaattgtcccttaaacaaaaattaaaatcctcaatccagctatgttatattgaaaaaatagagcctgagggatctttactagttataaagatacagaactctttcaaaaccttttgaaattaacctctcactataccagtataattgagttttcagtggggcagtcattatccaggtaatccaagatattttaaaatctgtcacgtagaacttggatgtacctgcccccaatccatgaaccaagaccattgaattcttggttgaggaaacaaacatgaccctaaatcttgactacagtcaggaaaggaatcatttctatttctcctccatgggagaaaatagataagagtagaaactgcagggaaaattatttgcataacaattcctctactaacaatcagctccttcctggagactgcccagctaaagcaatatgcatttaaatacagtcttccatttgcaagggaaaagtctcttgtaatccgaatctctttttgctttcgaactgctagtcaagtgcgtccacgagctgtttactagggatccctcatctgtccctccgggacctggtgctgcctctacctgacactcccttgggctccctgtaacctcttcagaggccctcgctgccagctctgtatcaggacccagaggaaggggccagaggctcgttgactggctgtgtgttgggattgagtctgtgccacgtgtatgtgctgtggtgtgtccccctctgtccaggcactgagataccagcgaggaggctccagagggcactctgcttgttattagagattacctcctgagaaaaaagcttccgcttggagcagaggggctgaatagcagaaggttgcacctcccccaaccttagatgttctaagtctttccattggatctcattggacccttccatggtgtgatcgtctgactggtgttatcaccgtgggctccctgactgggagttgatcgcctttcccaggtgctacacccttttccagctggatgagaatttgagtgctctgatccctctacagagcttccctgactcattctgaaggagccccattcctgggaaatattccctagaaacttccaaatcccctaagcagaccactgataaaaccatgtagaaaatttgttattttgcaacctcgctggactctcagtctctgagcagtgaatgattcagtgttaaatgtgatgaatactgtattttgtattgtttcaagtgcatctcccagataatgtgaaaatggtccaggagaaggccaattcctatacgcagcgtgctttaaaaaataaataagaaacaactctttgagaaacaacaatttctactttgaagtcataccaatgaaaaaatgtatatgcacttataattttcctaataaagttctgtactcaaatgta

SEQ ID NO: 8 - protein, 195 aa, Homo sapiens
MAWEARREPGPRRAAVRETVMLLLCLGVPTGRPYNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQGSISKYRART

SEQ ID NO: 9 - DNA, 1427 bp, Homo sapiens
gggggctcttgctgagcccagagccatcccgcgctctgcgggctgggaggcccgggccaggacgcgagtcctgcgcagccgaggttccccagcgccccctgcagccgcgcgtaggcagagacggagcccggccctgcgcctccgcaccacgcccgggaccccacccagcggcccgtacccggagaagcagcgcgagcacccgaagctcccggctggcggcagaaaccgggagtggggccgggcgagtgcgcggcatcccaggccggcccgaacgctccgcccgcggtgggccgacttcccctcctcttccctctctccttcctttagcccgctggcgccggacacgctgcgcctcatctcttggggcgttcttccccgttggccaaccgtcgcatcccgtgcaactttggggtagtggccgtttagtgttgaatgttccccaccgagagcgcatggcttgggaagcgaggcgcgaacccggcccccgaagggccgccgtccgggagacggtgatgctgttgctgtgcctgggggtcccgaccggccgcccctacaacgtggacactgagagcgcgctgctttaccagggcccccacaacacgctgttcggctactcggtcgtgctgcacagccacggggcgaaccgatggctcctagtgggtgcgcccactgccaactggctcgccaacgcttcagtgatcaatcccggggcgatttacagatgcaggatcggaaagaatcccggccagacgtgcgaacagctccagctgggtagccctaatggagaaccttgtggaaagacttgtttggaagagagagacaatcagtggttgggggtcacactttccagacagccaggagaaaatggatccatcgtgacttgtgggcatagatggaaaaatatattttacataaagaatgaaaataagctccccactggtggttgctatggagtgccccctgatttacgaacagaactgagtaaaagaatagctccgtgttatcaaggtagcatcagcaagtacagagctaggacataacagaagtgagcagaattggggtgaatgtcaacagagcatgtcagaggatatctgcagcaaaactaaaatccaacaatgcgtctttaggagctaagaaacatatgaatgcaaattcataattttctaaccaccctcactctcaaaaaaaaatccctcagcacgcaaagaaaaattttattggttggcaaaactttggccaagtatttgagttcaattatgggtgctaataagactttggtctttcttccttggaatgtgatgtcacagagcagcttatattacatatattcctattttgccttcggatacccaaaagataaaaaagtgaatattgattctcagttgcaataaaactcagtcttgatttctgcaaaaaaaaaaaaaaaa

SEQ ID NO: 10 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 11 - DNA, 1662 bp, Homo sapiens
ccgagggcctgagtgctccagtagccaccgcatctggagaaccagcggttaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaaaaaaaaaaaaaaa

(19) United States
(12) Patent Application Publication
(10) Pub. No.: US 2004/0132642 A1
(43) Pub. Date: Jul. 8, 2004

(54) Methods of inhibiting metastasis or growth of a tumor cell

(75) Inventor: Sam Hwang, Potomac, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Government of the U.S.A., represented by the Secretary, Dept. of Health & Human Services, Rockville, MD

(21) Appl. No.: 10/706,265

(22) Filed: Nov. 12, 2003

Related U.S. Application Data
(60) Provisional application No. 60/425,472, filed on Nov. 12, 2002.
(60) Provisional application No. 60/511,581, filed on Oct. 15, 2003.

(51) Int. Cl. A61K 38/12

(52) U.S. Cl. 514/9; 514/10

(57) ABSTRACT

Methods of inhibiting metastasis of a CXCR4-expressing tumor cell in a mammal, which methods comprise administering to the mammal in an amount sufficient to inhibit metastasis of the tumor cell an antagonist of CXCR4, such as a polypeptide of the formula described herein, an antagonist of β₁ integrin, such as an antibody that binds to β₁, an antagonist of CXCL12, such as an antibody that binds to CXCL12, or an antagonist of α₄ integrin, such as an antibody that binds to α₄; and a method of inhibiting growth of a CXCR4 expressing-tumor cell in a mammal, which method comprises administering to the tumor cell a polypeptide of the formula described herein.

### CROSS-REFERENCE TO RELATED PATENT APPLICATIONS

This patent application claims the benefit of U.S. Provisional Patent Application No. 60/425,472, filed Nov. 12, 2002, and U.S. Provisional Patent Application No. 60/511,581, filed Oct. 15, 2003.

### FIELD OF THE INVENTION

The present invention pertains to methods of inhibiting metastasis of a CXC Chemokine Receptor-4 (CXCR4)-expressing tumor cell. The method comprises administering an antagonist of CXCR4, such as a polypeptide of the formula described herein, or an antagonist of β₁ integrin, CXCL12, or α₄ integrin in an amount sufficient to inhibit metastasis. The present invention also pertains to a method of inhibiting growth of a CXCR4-expressing tumor cell. The method comprises administering an antagonist of CXCR4, such as a polypeptide of the formula described herein, in an amount sufficient to inhibit growth of a tumor cell.

### BACKGROUND OF THE INVENTION

The metastasis of tumor cells represents the primary source of clinical morbidity and mortality in the large majority of solid tumors. Metastasis of cancer cells can result from detachment of tumor cells from the primary tumor and entry of tumor cells into either lymphatic or blood vessels. Invasion of lymphatic vessels results in metastasis to regional draining lymph nodes (LN). For many tumors, including melanoma, the presence of regional LN metastases is the single best predictor of patient survival (Coit et al., Ann Surg. 214: 627-36 (1991)). Invasion of veins or capillaries may result in the distribution of tumor cells to many body sites, but it is well-known that some locations are much more likely to be sites of tumor metastasis than others (Hart et al., Cancer Res. 40: 2281-7 (1980)). Melanoma, for example, tends to metastasize to the lung, liver, and brain (Cohn-Cedermark et al., Acta Oncl., 38: 549-57 (1999)). The mechanisms underlying organ-selective metastasis are still under intense investigation (Woodhouse et al., Cancer 80: 1529-37 (1997)).

Metastasis is a complex event (Engers et al., J. Cancer Res. Clin. Oncol. 126: 682-692 (2000)) with many factors that can potentially affect tumor distribution, such as the properties of the tumor cells and the microenvironment to which the tumor cells ultimately localize. There is emerging data suggesting that chemokines and their receptors may play key roles in determining the site of metastasis (Müller et al., Nature 410: 50-6 (2001); Geminder et al., J. Immunol. 167: 4747-57 (2001); Robledo et al., J Biol. Chem. 276: 45098-45105 (2001); and Taichman et al., Cancer Res. 62: 1832-7 (2002); International Patent Applications WO 99/47158 and WO 99/50461; and (Koshiba et al., Clin. Cancer Res. 6: 3530-3535 (2000)).

Chemokine receptors comprise four homologous families of 7-transmembrane-spanning, G-protein coupled receptors that activate key intracellular signaling pathways controlling cell shape, migration (chemotaxis), and proliferation (Locati et al., Annu. Rev. Med. 50: 425-440, (1999)). One of their functions is to increase leukocyte integrin affinity and avidity (Constantin et al., Immunity 13: 759-769 (2000)), leading to firm adhesion of leukocytes to vascular endothelium at sites of inflammation (Wong et al., Semin. Immunol. 15: 5-14 (2003)).

Integrins also appear to play important roles in melanoma invasion and metastasis through enhancement of motility and migration (Seftor et al., Cancer Metastasis Rev. 18:359-375 (1999); Menter et al., Immunol. Cell Biol., 73: 575-583 (1995); and Giavazzi et al., J. Clin. Invest. 92: 3038-3044 (1993)). Because integrins constitutively exhibit low affinity for their ligands, other cell-surface receptors, such as chemokine receptors, must usually be engaged first in order to increase the affinity, as well as avidity, of integrins for their ligands (Constantin et al., Immunity, 13:759-769 (2000); and Maki et al., J. Immunol. 169: 2346-2353 (2002)).

Despite static assays (Robledo et al., J. Biol. Chem. 276: 45098-45102 (2001); Libura et al., Blood 100: 2597-2606 (2002); Kijima et al., Cancer Res. 62: 6304-6311 (2002); and Murakami et al., Cancer Res. 62: 7328-7334 (2002)), the mechanism by which chemokines enhance tumor metastasis remains unclear, inasmuch as tumor cells must be able to adhere to endothelial cells in vivo in spite of the shear force exerted by vascular blood flow. To date, the ability of chemokines to trigger adhesion of cancer cells to endothelial cells under shear stress conditions has not been investigated.

In view of the foregoing, there is a need in the art for the identification of specific molecular factors involved in triggering adhesion of tumors cells to endothelial cells under shear stress conditions, such that methods of inhibiting metastasis of a tumor cell in vivo can be elucidated. The present invention identifies such molecular factors and provides such methods of inhibiting metastasis of a tumor cell. These and other objects and advantages of the invention, as well as additional inventive features, will be apparent from the description of the invention provided herein.

### BRIEF SUMMARY OF THE INVENTION

The present invention provides methods of inhibiting metastasis of a tumor cell in a mammal, wherein the tumor cell expresses a CXCR4. One of the methods comprises administering to the mammal in an amount sufficient to inhibit metastasis of the tumor cell a polypeptide of the formula:

Xaa₁-Xaa₂-Xaa₃Cys₄-Xaa₅-Xaa₆-Xaa₇-Cys₈-Xaa₉-Xaa₁₀-Gly₁₁-Xaa₁₂-Cys₁₃-Xaa₁₄-Xaa₁₅-Xaa₁₆-Cys₁₇-Xaa₁₈-Xaa₁₉-R (SEQ ID NO: 1)

wherein, each of Xaa₁, Xaa₂, and Xaa₁₉ is optionally a part of the polypeptide,

wherein, when Xaa₁ is a part of the polypeptide, Xaa₂ is part of the polypeptide,

wherein each of Xaa₃, Xaa₅, Xaa₉, Xaa₁₂, and Xaa₁₄ is an amino acid selected from the group consisting of Tyr, Phe, and Trp,

wherein each of Xaa₁, Xaa₂, Xaa₆, Xaa₇, Xaa₁₀, Xaa₁₅, Xaa₁₆, Xaa₁₈, and Xaa₁₉ is an amino acid selected from the group consisting of Arg and Lys,

wherein R is —OH or —NH₂,

wherein Cys₄ is optionally disulfide bonded to Cys₁₇, and

wherein Cys₈ is optionally disulfide bonded to Cys₁₃.

Another method of inhibiting metastasis of a tumor cell in a mammal provided herein comprises administering to the mammal in an amount sufficient to inhibit metastasis of the tumor cell an antagonist of CXCR4, wherein the antagonist is not an antibody that binds to CXCR4.

Yet another method of inhibiting metastasis of a tumor cell in a mammal provided by the present invention comprises administering to the mammal in an amount sufficient to inhibit metastasis of the tumor cell an antagonist of β₁ integrin, an antagonist of CXCL12, or an antagonist of α₄ integrin.

Further provided is a method of inhibiting growth of a tumor cell, wherein the tumor cell expresses CXCR4 and the growth is stimulated by SDF-1. The method comprises administering to the tumor cell in an amount sufficient to inhibit the growth of the tumor cell a polypeptide of the formula described above.

### BRIEF DESCRIPTION OF THE DRAWING

FIG. 1 sets forth the sequences discussed herein.

### DETAILED DESCRIPTION OF THE INVENTION

The present invention provides a method of inhibiting metastasis of a tumor cell in a mammal, wherein the tumor cell expresses a CXCR4. The method comprises administering to the mammal in an amount sufficient to inhibit metastasis of the tumor cell a polypeptide of the formula:

Xaa₁-Xaa₂-Xaa₃Cys₄-Xaa₅-Xaa₆-Xaa₇-Cys₈-Xaa₉-Xaa₁₀-Gly₁₁-Xaa₁₂-Cys₁₃-Xaa₁₄-Xaa₁₅-Xaa₁₆-Cys₁₇-Xaa₁₈-Xaa₁₉-R (SEQ ID NO: 1)

wherein, each of Xaa₁, Xaa₂, and Xaa₁₉ is optionally a part of the polypeptide,

wherein, when Xaa₁ is a part of the polypeptide, Xaa₂ is part of the polypeptide,

wherein each of Xaa₃, Xaa₅, Xaa₉, Xaa₁₂, and Xaa₁₄ is an amino acid selected from the group consisting of Tyr, Phe, and Trp,

wherein each of Xaa₁, Xaa₂, Xaa₆, Xaa₇, Xaa₁₀, Xaa₁₅, Xaa₁₆, Xaa₁₈, and Xaa₁₉ is an amino acid selected from the group consisting of Arg and Lys,

wherein R is —OH or —NH₂, and

wherein Cys₄ is optionally disulfide bonded to Cys₁₇, and

wherein Cys₈ is optionally disulfide bonded to Cys₁₃.

The present invention further provides a method of inhibiting growth of a tumor cell, wherein the tumor cell expresses CXCR4 and the growth is stimulated by SDF-1. The method comprises administering to the tumor cell in an amount sufficient to inhibit the growth of the tumor cell a polypeptide of the above-described formula.

For purposes of the present invention, any polypeptide can be used in the present inventive methods, provided that it is a polypeptide of the above-defined formula. Preferably, the polypeptide is Arg-Arg Trp-Cys-Tyr-Arg-Lys-Cys-Tyr-Lys-Gly-Tyr-Cys-Tyr-Arg-Lys-Cys-Arg (SEQ ID NO: 2), in which the first and second Cys residues are disulfide bonded to the fourth and third Cys residues, respectively. This polypeptide is known in the art as T22.

Suitable methods of synthesizing the polypeptides of the formula described herein are known in the art and are described herein and in references, such as U.S. Pat. No. 5,449,752. Alternatively, the polypeptides described herein can be commercially synthesized by companies, such as Synpep (Dublin, Calif.).

The present invention further provides another method of inhibiting metastasis of a tumor cell in a mammal, wherein the tumor cell expresses CXCR4. This method comprises administering to the mammal an antagonist of CXCR4, which is not an antibody that binds to CXCR4, in an amount sufficient to inhibit metastasis of the tumor cell. The term “antagonist” as used herein refers to any substance that counteracts the cellular effects of a natural compound (such as a hormone or neurotransmitter) by binding to the cellular receptor for the compound and blocking its action or by binding to the natural compound and blocking its action. Antagonists suitable for use in the present inventive methods include, for example, AMD-3100, T134, ALC40-4C, CGP64222, and HIV-1 Pat Protein, of which AMD-3100 is a small molecule CXCR4 antagonist described in Hendrix et al., Antimicrob. Agents Chemother. 44: 1667-1673 (2000), and of which T134, ALX40-4C, T140, and CGP64222 are 9 amino acid-polypeptides described in Arakaki et al., J. Virol. 73: 1719-1723 (1999); Doranz et al., AIDS Res. Hum. Retroviruses 17: 475-486 (2001); Tamamura et al., Biochem. Biophys. Res. Commun. 253: 877-882 (1998); and Ed Clercq et al., Antivir. Chem. Chemother. 12: 19-31 (2001).

Also provided by the present invention is yet another method of inhibiting metastasis of a tumor cell in a mammal, wherein the tumor cell expresses CXCR4. This method comprises administering to the mammal in an amount sufficient to inhibit metastasis of the tumor cell an antagonist of β₁ integrin. β₁ integrin is an adhesion molecule involved in tumor migration. This molecule plays an important role in melanoma invasion and metastasis through enhancement of motility and migration (Seyton et al., Cancer Metastasis Review, 18:359-375 (1999)).

Suitable antagonists of β₁ integrin are known in the art, and include, for instance, an antibody that binds to β₁ integrin, a peptide or peptidomimetic agent that binds to β₁ integrin or that binds to the ligand of β₁ integrin, and a soluble β₁ integrin. Ligands of β₁ integrin include fibronectin, laminin, and (vascular cell adhesion molecule-1) VCAM-1 (Shimaoka and Springer, Nat. Rev. Drug Discovery 2: 703-716 (2003)). The peptide or peptidomimetic agent that binds to β₁ integrin can be, for instance, a peptide or peptidomimetic agent that comprises, consists essentially of, or consists of the receptor-binding portion of a ligand for β₁ integrin. For instance, such peptides or peptidomimetic agent can have the amino acid sequence RGD. The peptide or peptidomimetic agent that binds to the ligand of β₁ integrin can be, for example, a peptide or peptidomimetic agent comprising, consisting essentially of, or consisting of the ligand-binding portion of the β₁ integrin. The amino acid and nucleotide sequences of the human β₁ integrin are set forth below as SEQ ID NOs: 4 and 5, respectively. The soluble β₁ integrin can be, for instance, a β₁ integrin protein lacking the transmembrane domain of the β₁ integrin protein. In this respect, the ligand binds to the soluble β₁ integrin protein but does not mediate intercellular contact. Preferably, the antagonist of β₁ integrin is an antibody that binds to β₁ integrin, i.e., an antibody that specifically reacts with or recognizes β₁ integrin. Desirably, the antibody that binds to β₁ integrin is a blocking antibody, i.e., a neutralizing antibody; one that binds and does not elicit or induce downstream intracellular signaling and that blocks or prevents other ligands or agents from binding to the β₁ integrin. Such antibodies are publicly available from companies, such as BD-Pharmingen (San Diego, Calif.).

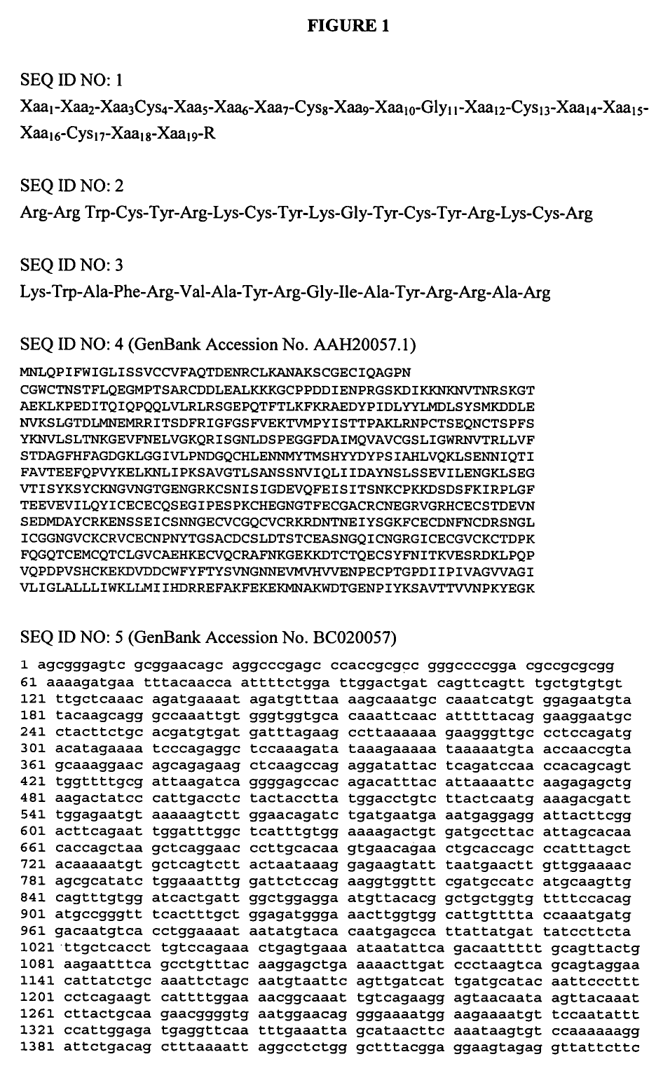

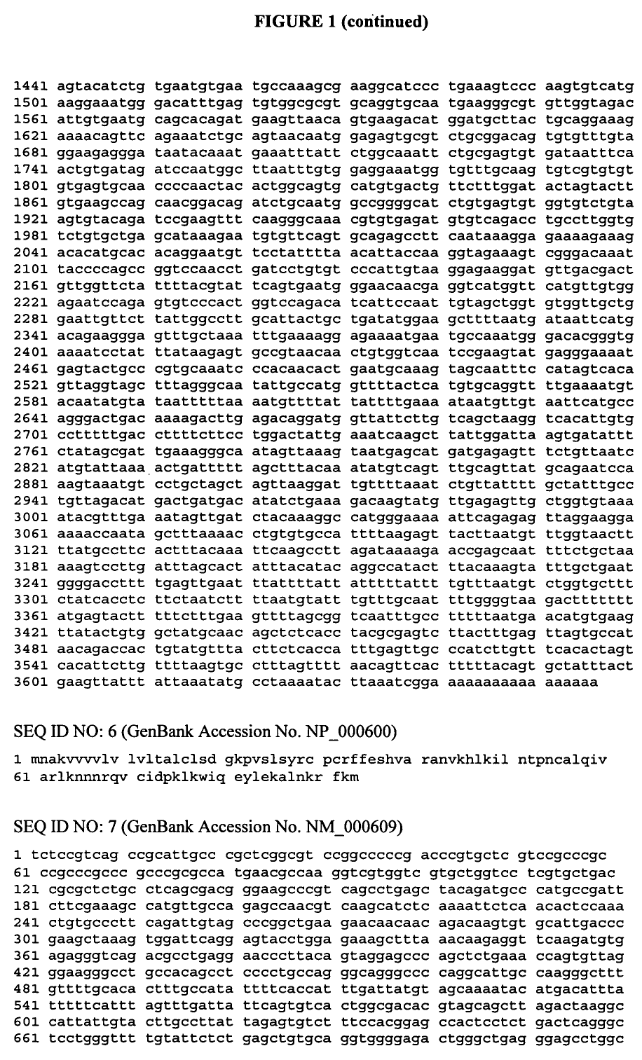

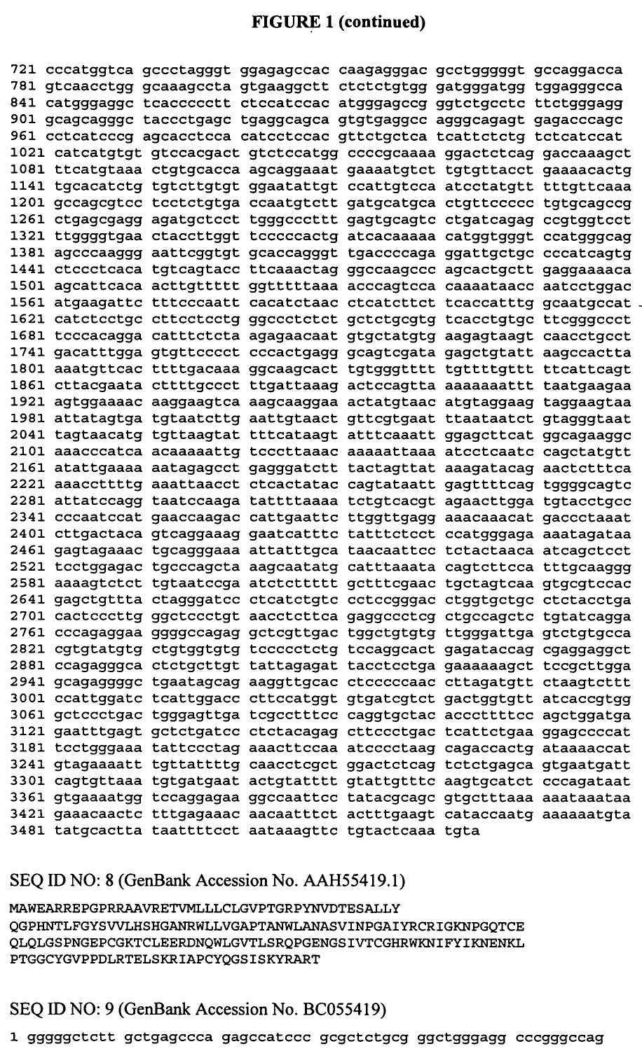

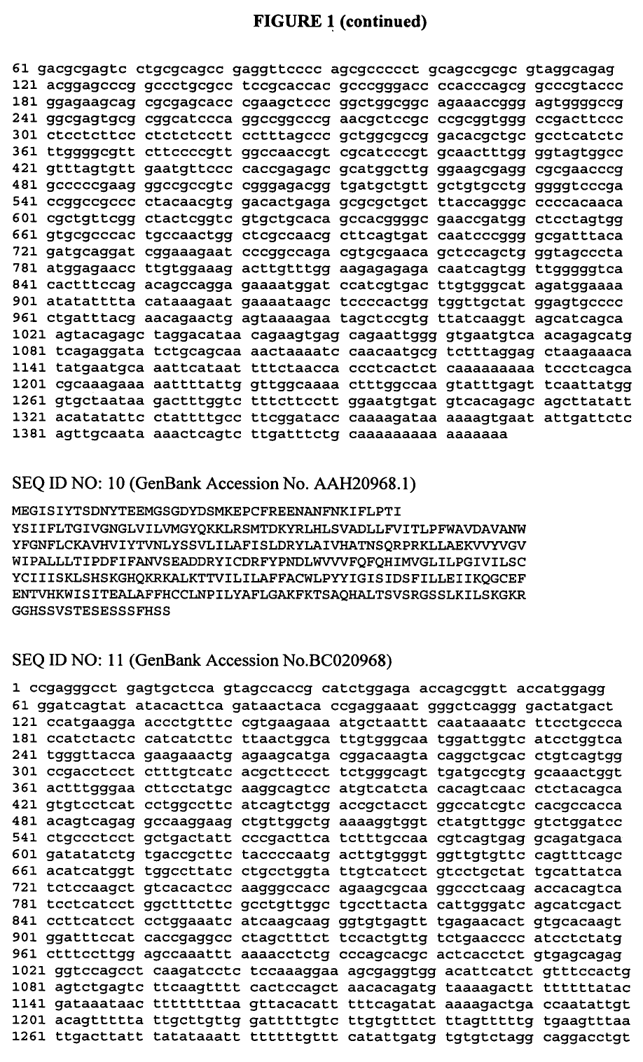

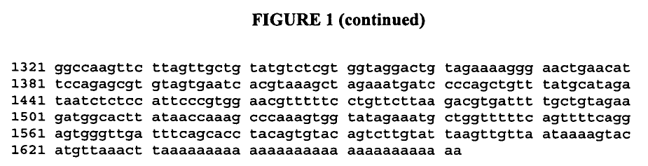

The present invention provides yet another method of inhibiting metastasis of a tumor cell in a mammal, wherein the tumor cell expresses CXCR4. This method comprises administering to the mammal in an amount sufficient to inhibit metastasis of the tumor cell an antagonist of CXCL12. Suitable antagonists of CXCL12 for use in the present inventive method are known in the art and include, for instance, an antibody that binds to CXCL12; a peptide or peptidomimetic agent that binds to CXCL12 or that binds to the receptor of CXCL12, i.e., CXCR4; and a soluble CXCR4. The peptide or peptidomimetic agent that binds to CXCL12 can be, for instance, a peptide or peptidomimetic agent that comprises, consists essentially of, or consists of the ligand-binding portion of CXCR4. The amino acid and nucleotide sequences of human CXCR4 are set forth in FIG. 1 as SEQ ID NOs: 10 and 11, respectively. The peptide or peptidomimetic agent that binds to the receptor of CXCL12 can be, for example, a peptide or peptidomimetic agent comprising, consisting essentially of, or consisting of the receptor-binding portion of the CXCL12. The amino acid and nucleotide sequences of human CXCL12 are set forth in FIG. 1 as SEQ ID NOs: 6 and 7, respectively. The soluble CXCR4 can be, for instance, a CXCR4 protein lacking the transmembrane domain of the CXCR4 protein. In this respect, CXCL12 binds to the soluble CXCR4 protein but does not mediate intracellular signaling or intercellular contact. Preferably, the antagonist of CXCL12 is an antibody that binds to CXCL12. Desirably, the antibody that binds to CXCL12 is a blocking antibody. Such antibodies are publicly available from companies, such as R & D Systems (Minneapolis, Minn.).

The present invention provides another method of inhibiting metastasis of a tumor cell in a mammal, wherein the tumor cell expresses CXCR4. The method comprises administering to the mammal in an amount sufficient to inhibit metastasis an antagonist of α₄ integrin. Suitable antagonists of α₄ integrin for use in the present inventive methods include, for instance, an antibody that binds to α₄ integrin, a peptide or peptidomimetic agent that binds to α₄ integrin or that binds to the ligand of α₄ integrin, and a soluble α₄ integrin. The peptide or peptidomimetic agent that binds to α₄ integrin can be, for instance, a peptide or peptidomimetic agent that comprises, consists essentially of, or consists of the receptor-binding portion of a ligand of α₄ integrin. The peptide or peptidomimetic agent that binds to the ligand of α₄ integrin can be, for example, a peptide or peptidomimetic agent comprising, consisting essentially of, or consisting of the ligand-binding portion of the α₄ integrin. The amino acid and nucleotide sequences of human α₄ integrin are set forth in FIG. 1 as SEQ ID NOs: 8 and 9, respectively. The soluble α₄ integrin can be, for instance, an α₄ integrin protein lacking the transmembrane domain of the α₄ integrin protein. In this respect, the ligand binds to the soluble α₄ integrin protein but does not mediate intercellular contact. Preferably, the antagonist of α₄ is an antibody that binds to α₄ integrin. Desirably, the antibody that binds to α₄ integrin is a blocking antibody. Such antibodies are publicly available from companies, such as eBioscience (San Diego, Calif.) and BD-Pharmingen (Sand Jose, Calif.).

For purposes of the inventive methods described herein, the tumor cell can be a tumor cell that can metastasize to an organ, which comprises cells that express SDF-1. Such organs include, but are not limited to, the skin, lung, liver, and brain. It is most preferred that the organ is the lung. In this regard, the metastasis to be inhibited by the present inventive methods is preferably the metastasis to the skin, lung, liver, or brain.

The tumor cell of the present inventive methods can be any tumor cell or cancer cell, provided that the cell expresses CXCR4. Such cells include, but are not limited to, a melanoma cell, a breast cancer cell, a breast tumor cell, a lymphoma cell, a neuroblastoma cell, a lung cancer cell, an angiosarcoma cell, a pancreatic cancer, a leukemia cell, and a prostate cancer cell. Preferably, the tumor cell is a melanoma cell.

Suitable methods of determining whether or not a cell expresses a particular protein are known in the art (see, for instance, Sambrook et al., Molecular Cloning: A Laboratory Manual, 2^{nd} Ed., Cold Spring Harbor, N.Y., 1989). With regards to the present inventive methods, whether or not a tumor cell expresses CXCR4 or whether or not a cell expresses SDF-1 can be determined by, for example, Fluorescence-Activated Cell Sorting (FACS) using fluorescein isothiocyante (FITC)-conjugated antibodies, which recognize either CXCR4 or SDF-1. Western blotting or immunofluorescence also could be performed to determine whether or not a cell expresses SDF-1 or a tumor cell expresses CXCR4. These methods are well-known in the art.

Methods of determining whether or not growth of a tumor cell is stimulated by SDF-1 are also known in the art. One suitable method is described herein as Example 5.

The polypeptide or antagonist of the present inventive methods can be in the form of a salt, which is preferably a pharmaceutically acceptable salt. Suitable pharmaceutically acceptable acid-addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example p-toluenesulphonic acid.

With respect to the present inventive methods, the polypeptide of the formula described herein, as well as the antagonist of CXCR4, β₁ integrin, CXCL12, or α₄ integrin can be a part of a composition, such as a pharmaceutical composition. The pharmaceutical composition can comprise more than one active ingredient, such as more than one polypeptide antagonist, β₁ integrin antagonist, CXCL12 antagonist, or α₄ integrin antagonist. The pharmaceutical composition can alternatively comprise one or more of the polypeptides and/or one or more of the antagonists, alone or in combination with another pharmaceutically active agent or drug.

The composition can further comprise a carrier. The carrier can be any suitable carrier. Preferably, the carrier is a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. It will be appreciated by one of ordinary skill in the art that, in addition to the following described pharmaceutical compositions, the polypeptides or any of the antagonists of the present inventive methods can be formulated as inclusion complexes, such as cyclodextrin inclusion complexes, or liposomes.

The pharmaceutically acceptable carriers described herein, for example, vehicles, adjuvants, excipients, and diluents, are well-known to those ordinarily skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular polypeptide or antagonist, as well as by the particular method used to administer the polypeptide or antagonist. Accordingly, there are a variety of suitable formulations of the pharmaceutical composition of the present inventive methods. The following formulations for oral, aerosol, parenteral, subcutaneous, intravenous, intramuscular, interperitoneal, rectal, and vaginal administration are exemplary and are in no way limiting. One ordinarily skilled in the art will appreciate that these routes of administering the polypeptide or any of the antagonists of the present invention are known, and, although more than one route can be used to administer the polypeptide, a particular route can provide a more immediate and more effective response than another route.

Injectable formulations are among those formulations that are preferred in accordance with the present invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed., pages 622-630 (1986)).

Topical formulations are also well-known to those of ordinary skill in the art. Such formulations are particularly suitable in the context of the present invention for application to the skin.

Formulations suitable for oral administration can consist of (a) liquid solutions, such as an effective amount of the inhibitor dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The polypeptide of the formula described herein, CXCR4 antagonist, β₁ antagonist, CXCL12 antagonist, or α₄ integrin antagonist, alone or in combination with each other and/or with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The polypeptide or antagonists of the present invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

The parenteral formulations will typically contain from about 0.5% to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Additionally, the polypeptides of the formula described herein, CXCR4 antagonists, β₁ integrin antagonists, CXCL12 antagonists, or α₄ integrin antagonists, or compositions comprising any of the foregoing, can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

Preferably, the polypeptides of the formula described herein are administered to the mammal intraperitoneally.

One of ordinary skill in the art will readily appreciate that the polypeptides or antagonists of the present inventive methods can be modified in any number of ways, such that the therapeutic efficacy of the polypeptide or antagonist is increased through the modification. For instance, the polypeptide or antagonist could be conjugated either directly or indirectly through a linker to a targeting moiety. The practice of conjugating compounds, such as polypeptides and antagonists, to targeting moieties is known in the art. See, for instance, Wadwa et al., J. Drug Targeting 3: 111 (1995), and U.S. Pat. No. 5,087,616. The term “targeting moiety” as used herein, refers to any molecule or agent that specifically recognizes and binds to a cell-surface receptor, such that the targeting moiety directs the delivery of the polypeptide or antagonist to a population of cells on which surface the receptor is expressed. Targeting moieties include, but are not limited to, antibodies, or fragments thereof, peptides, hormones, growth factors, cytokines, and any other naturally- or non-naturally-existing ligands, which bind to cell surface receptors. The term “linker” as used herein, refers to any agent or molecule that bridges the polypeptide or antagonist to the targeting moiety. One of ordinary skill in the art recognizes that sites on the polypeptide or antagonist, which are not necessary for the function of the polypeptide or antagonist, are ideal sites for attaching a linker and/or a targeting moiety, provided that the linker and/or targeting moiety, once attached to the polypeptide or antagonist, do(es) not interfere with the function of the polypeptide or antagonist, i.e., the ability to inhibit metastasis or growth of a tumor cell in a mammal.

Alternatively, the polypeptides or antagonists of the present invention can be modified into a depot form, such that the manner in which the polypeptide or antagonist is released into the body to which it is administered is controlled with respect to time and location within the body (see, for example, U.S. Pat. No. 4,450,150). Depot forms of polypeptides or antagonists can be, for example, an implantable composition comprising the polypeptide or antagonist and a porous material, such as a polymer, wherein the polypeptide or antagonist is encapsulated by or diffused throughout the porous material. The depot is then implanted into the desired location within the body and the polypeptide or antagonist is released from the implant at a predetermined rate by diffusing through the porous material.

Furthermore, the present inventive methods can comprise the administration of the polypeptide or antagonist, in the presence or absence of an agent that enhances its efficacy, or the methods can further comprise the administration of other suitable components, such as anti-angiogenic agents and chemotherapeutic agents, including cisplatin, doxirubicin, daunorubicin, and camphotecin.

For purposes of the present inventive method, the amount or dose of the polypeptide (of the formula described herein) or any of the antagonists (described herein) administered should be sufficient to effect a therapeutic response in the animal over a reasonable time frame. Particularly, the dose of the polypeptide or antagonist should be sufficient to inhibit metastasis of a tumor cell in a mammal within about 2 days, if not 14 days, from the time of administration. Also preferred is that the dose of the antagonist is sufficient to inhibit metastasis of a tumor cell or that the dose of the polypeptide of the present invention is sufficient to inhibit tumor cell growth. Desirably, the polypeptides of the formula described herein are administered to the mammal daily for at least 2 days. The dose will be determined by the efficacy of the particular polypeptide or antagonist and the condition of the animal (e.g., human), as well as the body weight of the animal (e.g., human) to be treated. Many assays for determining an administered dose are known in the art. For purposes of the present invention, an assay, which comprises comparing the extent to which metastasis or growth of a tumor cell is inhibited upon administration of a given dose of a polypeptide of the formula described herein or a CXCR4 antagonist to a mammal among a set of mammals, each of which is given a different dose of the polypeptide, could be used to determine a starting dose to be administered to a mammal. The extent to which metastasis of a tumor cell is inhibited upon administration of a certain dose can be assayed as described herein as Example 3, while the extent to which growth of a tumor cell is inhibited upon administration of a certain dose can be assayed as described herein as Example 5.

The size of the dose also will be determined by the existence, nature and extent of any adverse side effects that might accompany the administration of a particular polypeptide or CXCR4 antagonist. Ultimately, the attending physician will decide the dosage of the polypeptide or antagonist of the present invention with which to treat each individual patient, taking into consideration a variety of factors, such as age, body weight, general health, diet, sex, inhibitor to be administered, route of administration, and the severity of the condition being treated.

For purposes of the present invention, mammals include, but are not limited to, the order Rodentia, such as mice, and the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

As used herein, the terms “inhibits” and “blocks” and words stemming from either of the foregoing, do not necessarily imply a 100% or complete inhibition or blockage. Rather, there are varying degrees of inhibition or blockage of which one of ordinary skill in the art recognizes as having a potential benefit or therapeutic effect. In this regard, the polypeptide of the formula described herein or the antagonist can achieve any level of inhibition or blockage of tumor cell metastasis or any level of inhibition of tumor cell growth. Desirably, the polypeptide or antagonist inhibits or blocks at least 10% of the tumor cell metastasis or growth, which occurs in the absence of any polypeptide of the formula described herein or antagonist. It is more preferred that the polypeptide of the formula herein or antagonist achieves at least about 50% inhibition. Most preferred is that the polypeptide or antagonist inhibits at least about 90% of the tumor cell metastasis or tumor cell growth that occurs in the absence of any polypeptide of the formula described herein or antagonist.

### EXAMPLES

Abbreviations

For convenience, the following abbreviations are used herein: CXCR4, CXC Chemokine Receptor-4; LN, lymph nodes; CCR7, CC Chemokine Receptor 7; CCR10, CC Chemokine Receptor 10; CXCL12, CXC Ligand 12; SDF-1, Stromal Derived Factor-1; T22, [Tyr^{5, 12}, Lys⁷]-polyphemusin II; HIV-1, Human Immunodeficiency Virus-1; FACS, fluorescence-activated cell sorting; FITC, fluorescein isothiocyanate; HLB, hydrophile-lipophile balance; NCI, National Cancer Institute; NCI-FCRDC, National Cancer Institute-Frederick Cancer Research and Development Center; DMEM, Dulbecco's modified Eagle's medium; FCS, Fetal Calf Serum; DMEC, Dermal microvascular endothelial cells; LMEC, Lung microvascular endothelial cells; ALA, NH2-KWAFRVAYRGIAYRRAR-COOH; MEC, microvascular endothelial cells; and PTX, pertussis toxin.

Materials

The following describes the animals, cell lines, and reagents used in Examples 1-6 described herein: Female B57BL/6 mice (8-12 weeks old) from NCI-FCRDC, (Frederick, Md.) were used in all experiments, which have been approved by the NCI Animal Use and Care Committee. Syngeneic B16/F1 melanoma cells (Fidler, Nature (New Biol.) 242: 148-9 (1973)) were provided by NCI-FCRDC and grown in DMEM, (Gibco, Gaithersburg, Md.) with 10% heat-inactivated FCS and supplements (Wiley et al., J. Natl. Cancer Inst. 93: 1638-43 (2001)). Human DMEC were derived from neonatal foreskin and cultured as previously described (Fitzhugh et al, J. Immunol. 165: 6677-81 (2000)). Human LMEC (Clonetics-Cambrex, E. Rutherford, N.J.) were grown in EGM™-2 MV as recommended by the supplier. The CXCR4 function-blocking peptide, T22 (NH₂-RRWCYRKCYKGYCYRKCR-COOH (SEQ ID NO: 2) with disulfide bridges between the first and last (as well as the second and third) cysteine residues) (Murakami et al., J. Exp. Med 186:1389-93 (1997)), and a control peptide of similar amino acid composition, 4Ala-T-I (ALA, NH₂-KWAFRVAYRGIAYRRAR-COOH) (SEQ ID NO: 3) (Murakami et al. (1997), supra), were synthesized by Synpep (Dublin, Calif.) and HPLC-purified to >92% purity. In control experiments, T22 (500 ng/ml) was shown to block specifically in vitro chemotaxis of activated bone-marrow-derived dendritic cells to 200 ng/ml of CXCL12, whereas ALA had no inhibitory effect.

Statistical Calculations

The following describes the statistical rules used in Examples 1-6 described herein: P values were based on two-sided, parametric Student t tests using the program Instat (Graphpad, San Diego, Calif.) with statistical significance set at P<0.05.

The following examples serve to illustrate the present invention but, of course, should not be construed as in any way limiting its scope.

### Example 1

This example demonstrates the retroviral transduction of B16/F1 melanoma cells and subsequent testing of these cells.

Human CXCR4 cDNA (Feng et al., Science 272: 872-7 (1996)) (gift from Dr. E. Berger, NIAID) was subcloned into the pLNCX2 retroviral vector (Clontech, Palo Alto, Calif.). Using this vector, B16/F1 melanoma cells were transduced using a method previously described (Wiley et al. (2001), supra) to yield CXCR4-B16 cells. Transduction efficiency varied from 40-80%. The chemokine receptor-transduced and the vector-transduced tumor lines were subsequently retrovirally transduced (using a puromycin-based selection system) with firefly (Photinus pyralis) luciferase cDNA that was originally PCR-amplified from a luciferase-containing expression vector (pGL3-luciferase, Promega, Madison, Wis.). For convenience, the CXCR4-luciferase B16 line is hereafter called CXCR4-B16, while the control vector-luciferase-transduced line is called pLNCX2-B16.

To obtain consistent levels of CXCR4 expression, CXCR4-B16 cells were stained with PE-conjugated anti-human CXCR4 mAb (clone 12G5, Pharmingen, San Diego, Calif.), treated with anti-PE MicroBeads (Miltenyi Biotec Inc., Auburn, Calif.), washed, and loaded onto a MACS MS column (Miltenyi) for positive magnet-based selection. The positive fraction was then cultured with G418 and puromycin to maintain expression of CXCR4 and luciferase, respectively.

Luciferase activity was measured using a Luciferase Reporter assay system (Promega, Madison, Wis.). Cultured cells were lysed with 100 μl of undiluted lysis buffer and then assayed in the presence of luciferin using a Thermo Labsystems MLX 96-well luminometer (Helsinki, Finland). Units are in arbitrary light units. For tissue assays, organs from each animal were homogenized in 1 ml of lysis buffer, of which 25 μl aliquots were then assayed in duplicate. Means of duplicates were used to represent the luciferase activity for a given tissue from a particular animal. Five to ten animals per experimental treatment group were used for typical experiments.

As shown previously, B16/F1 melanoma cells have little to no expression of chemokine receptors, including CCR7 and CXCR4 (Wiley et al. (2001), supra). Because mAbs were available that recognized human CXCR4 and because human and murine CXCL12 have nearly complete homology and are functionally equivalent, B16/F1 melanoma cells were transduced with human CXCR4 cDNA in order to evaluate the possible role of CXCR4 in melanoma metastasis. Although initial transduction efficiency was ˜50%, expression could be increased to >80% of B16 cells by the use of specific anti-human CXCR4 mAb followed by magnetic bead selection. After confirming that the CXCR4 receptor was functional by calcium flux assay (Tiffany et al., JEM 186: 165-170 (1997)), CXCR4-expressing B16 cells were transduced with cDNA for firefly luciferase in order to facilitate quantitative analysis of metastasis. The advantages of luciferase as a marker include its sensitivity (since as few as fifty luciferase-transduced cells could be detected over background in vitro) and its linear dose-dependent output of light in the presence of luciferin (Table 1). Luciferase activity per cell was comparable in the pLNCX2- and CXCR4-B16 cell lines. All subsequent experiments were performed with double-transduced cells.

TABLE 1

Luciferase activity (RLU)

| Cell number | pLNCX2 | CXCR4 |
|---|---|---|
| 0 | 5 ± 1 | 5 ± 1 |
| 100 | 763 ± 14 | 717 ± 10 |
| 1,000 | 6,396 ± 390 | 6,340 ± 103 |
| 10,000 | 67,295 ± 1021 | 61,596 ± 1,038 |
| 100,000 | 62,3364 ± 20779 | 57,6983 ± 7635 |

This example demonstrated the construction of cells used in the following examples.

### Example 2

This example demonstrates that the expression of CXCR4 increases pulmonary metastasis after intravenous and subcutaneous inoculation of B16 tumor cells.

CXCR4- and pLNCX2-B16 cells in exponential growth phase were harvested by trypsinization and washed twice in PBS prior to injection. Cell viability was >95% as determined by trypan blue dye exclusion. For the footpad (i.e., subcutaneous) injection, B16 cells (4×10⁵ cells in 20 μl PBS) were subcutaneously injected into the left footpads of C57BL/6 mice. Tumor growth was monitored three times per week by measurement of two maximum perpendicular tumor diameters. When tumors reached 5-7 mm in size, animals were euthanized prior to harvesting of lung and LN. Popliteal LN were removed by gently pulling skin (including the dermis and subcutaneous fat) above the popliteal fossa and carefully exposing the LN resting in the adipose tissue of the fossa with a pair of forceps. For intravenous injection, 4×10⁵ CXCR4- or pLNCX2-B16 cells in 200 μl were injected into the tail veins of mice. T22 and ALA peptides in sterile PBS were administered via an intraperitoneal (i.p.) route using 4 μg peptide/mouse daily for the indicated duration of treatment. Experiments were performed 2-4 times each with similar trends.

To assess the effect of CXCR4 on pulmonary metastasis, either CXCR4-B16 cells or pLNCX2-B16 cells were injected intravenously into syngeneic mice. Expression of CXCR4 resulted in a remarkable increase (˜10-fold by luciferase activity) in pulmonary accumulation of the B16 cells by 10 days. Although intravenous inoculation provides a convenient model in which to assay pulmonary metastases, its relevance to distant organ metastasis in animals and humans is limited. To determine the effect of CXCR4 expression on pulmonary metastasis in an orthotopic model, we injected either CXCR4- or pLNCX2-B16 cells into the footpads of mice and waited 18 days for tumors to grow in size. Footpad tumor sizes were not statistically different between the two groups (P>0.05, n=7-10). Pulmonary metastases, however, were significantly enhanced (˜10-fold, P=0.03) in the mice that had been injected with CXCR4-B16 cells in their footpads (Table 2). While significant differences between these two groups were readily apparent after quantification by luciferase assay, the gross appearance of the lungs of the two groups of mice revealed no obvious macroscopic tumor metastasis. The draining popliteal LN of both sets of mice was also examined by luciferase assay, and no statistically significant pattern of enhanced LN metastasis in CXCR4-B16-injected animals was found. Thus, CXCR4 significantly increased pulmonary metastasis in both i.v. and s.c. injection models, without enhancing LN metastasis.

TABLE 2

| Cells | luciferase activity (RLU) |
|---|---|
| pLNCX2 | 50.0 ± 42.5 |
| CXCR4 | 584.5 ± 718.4 |

To determine the kinetics of tumor accumulation after i.v. inoculation, tumor cells were injected, and the accumulation in the lungs at 2, 6, 24, and 48 hr following injection was assessed. Large numbers of cells were present to similar degrees in the lungs two hours after injection, but this was followed by a sharp decrease in tumor cells over the next 4 hours (Table 3). At the 24 hr time point, however, there were ˜5-fold more cells in CXCR4-B16-injected animals (P<0.05) and 10-fold more at the 48 hr time point (P=0.01).

TABLE 3

| | Luciferase activity (RLU) | | | | |
|---|---|---|---|---|---|
| Cells | at 0 h | at 2 h | at 6 h | at 24 h | at 48 h |
| CXCR4- | 0 | 5275 ± 931 | 1095 ± 336 | 549 ± 269 | 1176 ± 578 |
| B16 | | | | | |
| pLNCX2- | 0 | 6191 ± 828 | 559 ± 261 | 113 ± 21 | 132 ± 69 |
| B16 | | | | | |

This example demonstrated that, while CXCR4 expression did not lead to an increase in the number of accumulated tumor cells 2 or 6 hr after injection, significantly increased numbers of tumor cells were found in the lungs of mice injected with CXCR4-B16 cells 24 and 48 hr after initial i.v. inoculation.

### Example 3

This example demonstrates that T22 inhibits CXCR4-mediated increases in metastasis.

Samples of primary cutaneous human melanoma and pulmonary metastases were obtained with institutional approval, frozen in liquid nitrogen, sectioned (5 μm), and fixed in ice-cold acetone. Endogenous peroxidase activity was blocked by hydrogen peroxide (0.3%) for 30 min at RT. Sections were stained with anti-human CXCR4 mAb (4 ug/ml, R&D Systems, Minneapolis, Minn.) overnight at 4° C., a biotinylated anti-mouse secondary antibody, and a streptavidin-horseradish peroxidase complex (Vectastain ABC, Vector Labs, Burlingame, Calif.). 3-Amino-9-ethylcarbazole (AEC, Vector Labs) was used for color (red) visualization. Sections were counterstained with hematoxylin.

T22 is an eighteen-amino acid peptide that was first described to antagonize HIV-1 activity in vitro (Nakashima et al., Antimicrob Agents Chemother. 36: 1249-55 (1992)) and then shown to block specifically CXCR4-mediated HIV-1 fusion (Murakami et al. (1997), supra). To determine whether T22 would inhibit the CXCR4-mediated increase in metastasis, mice were injected intravenously with CXCR4-B16 and pLNCX2-B16 cells. Subgroups of CXCR4-B16 mice were treated daily with 4 μg of either T22 or ALA control peptide. CXCR4-B16 cells accumulated in greater numbers in the lung compared to pLNCX2-B16 cells (Table 4), confirming the results described in Example 2.

TABLE 4

| Injected cells | Treatment (I.P.) | Luciferase activity (RLU) |
|---|---|---|
| pLNCX2 | PBS | 592 ± 862 |
| CXCR4 | PBS | 30790 ± 22587 |
| CXCR4 | T22 | 1114 ± 956 |
| CXCR4 | ALA | 43928 ± 29729 |

Treatment of the CXCR4-B16-injected mice with T22 reduced metastasis to levels seen in pLNCX2-B16 injected mice, while treatment with ALA had no effect. Histological examination of the lungs of CXCR4-B16-injected mice revealed that the majority of the lung tissue was occupied by tumor, explaining the increased mass of the lungs (Table 5).

TABLE 5
Average weight of mice

| Mice | Average weight (g) |
|---|---|
| Normal Lung | 0.53 |
| pLNCX2 + PBS | 0.55 |
| CXCR4 + PBS | 0.85 |
| CXCR4 + T22 | 0.55 |
| CXCR4 + ALA | 0.88 |

In lungs of pLNCX2-B16-injected mice or T22-treated CXCR4-B16-injected mice, however, smaller tumor foci that left the pulmonary architecture largely intact were observed. T22 did not block metastasis in pLNCX2-B16-injected mice. Minimal levels of metastasis were detected in the livers, LN, and kidneys of pLNCX2- and CXCR4-B16 mice in several experiments. In one experiment, the mean luciferase levels for liver, kidney, and 4 pooled LN from pLNCX2-B16-injected mice were 26, 6, and 9 units, respectively, while activities in CXCR4-B16-injected mice were 10, 15, and 10 units, respectively. The background luciferase activity of lung tissue for non-injected, unmanipulated mice was 4 units. The low absolute value of luciferase activity (near the limits of detection) shown by organs other than the lungs render statistical calculations uninformative. Interestingly, T22 had no significant effect on the growth of CXCR4-B16 cells injected into the footpad of mice compared to either PBS or ALA.

This example illustrates that the CXCR4 antagonist, T22, blocks pulmonary metastasis, but not primary tumor growth, of CXCR4-B16 melanoma.

### Example 4

This example demonstrates that CXCL12 enhances adhesion of CXCR4-B16 cells to resting microvascular endothelial cells.

Prior to interaction with tumor cells, resting confluent DMEC or LMEC were exposed to 100 ng/ml of recombinant CXCL12 or CCL21 for 15 min at 20° C. as described (Cinamon et al., Nat. Immunol. 2: 515-22 (2001)). After unbound chemokine was washed away, calcein-AM-labeled (Molecular Probes, Eugene, Oreg.) CXCR4- or pLNCX2-B16 cells (2.5×10⁵/ml in B16 medium) were injected at 1.5 dynes/cm² into a parallel plate flow chamber (Glycotech, Gaithersburg, Md.) containing confluent DMEC or LMEC. In some cases, tumor cells were treated with pertussis toxin (PTX, Calbiochem, San Diego, Calif.) at 100 ng/ml for 2 hours at 37° C. to block Gᵢ-protein-coupled signaling. After two min, flow was stopped, which enabled B16 cells to interact with MEC in the absence of shear stress. After 5 min, shear was re-established at 1.5 dynes/cm² for up to 2 min in order to detach unbound cells; adherent cells were digitally photographed in 5-6 random fields (each field=1.18 mm²) with excitation at 488 nm and emission at 513 nm using sufficient exposure time (˜1 s) to allow distinction between non-moving and moving cells (which appeared as dim streaks) (Fitzhugh et al. (2000), supra). Cells were then counted with the software program IPlab (Scanalytics, Fairfax, Va.).

To determine whether enhanced adhesion might account for enhanced metastasis in CXCR4-B16 cells, the ability of recombinant CXCL12 to induce arrest of CXCR4-B16 cells to resting human DMEC derived from neonatal foreskin or to LMEC, which are more likely to be similar to pulmonary endothelial cells in vivo, was tested. Endothelial cells rapidly capture certain chemokines, including CXCL12, from exogenous sources (Cinamon et al. (2001), supra). Furthermore, CXCL12 has been shown to be expressed in situ by microvascular endothelial cells in the lung and skin (Salvucci et al., Blood 99: 2703-11 (2002)). DMEC and LMEC were exposed to recombinant chemokines. pLNCX2- or CXCR4-B16 cells were allowed to make contact with the MEC in the absence of shear forces. Wall shear stress was increased to remove unbound melanoma cells. Exogenously added CXCL12 was able to stimulate adhesion of CXCR4-B16 cells to DMEC by ˜5-fold, whereas CCL21 had no effect (Table 6). Interestingly, pLNCX2-B16 cells showed little appreciable adhesion to DMEC either with (Table 6) or without CXCL12 (Table 6).

TABLE 6

| Cells | No SDF | CCL19 | CXCL12 |
|---|---|---|---|
| B16-CXCR4 | 16 | 6.8 | 85.2 |
| B16-plncx2 | ND | ND | 0 |

With LMEC, it was observed that CXCR4-B16 cells remained associated to the endothelial cells, despite vigorous washing, and bound 2.5 times as efficiently to LMEC as did the control (pLNCX2-B16) cells (Table 7). In contrast to DMEC binding, exogenous pretreatment of LMEC with CXCL12 did not further increase CXCR4-B16 binding. Pretreatment of the CXCR4-B16 cells with PTX to block chemokine-mediated signaling resulted in decreasing binding to the level observed with pLNCX2-B16 cells (Table 7).

TABLE 7

| Cells | no SDF | with SDF |
|---|---|---|
| B16-CXCR4 | 277.333 | 320.8333 |
| B16-plncx2 | 121.2 | 88.8333 |
| B16-CXCR4-pertussis | 111.1667 | 126 |

This example demonstrated that the expression of CXCR4 increased adhesion of B16 cells to DMEC as well as LMEC.

### Example 5

This example demonstrates that CXCL12 enhances CXCR4-B16 growth under low serum conditions in vitro.

CXCR4- and pLNCX2-B16 cells (2×10⁴ cells/well) were cultured in triplicate for 24 hr in 24-well plates containing B16 growth medium (DMEM, 10% FCS). After rinsing the cells in serum-free DMEM, the B16 cells were incubated for 40 hr at 37° C. in DMEM containing low serum (0.5% FCS). At this point, CXCL12 (500 ng/ml) was added to the cells by itself or with either T22 or control ALA peptide at 1 μg/ml for the indicated times. Cells were then harvested at 24 or 48 hr later and counted by a hemacytometer.

In the absence of fetal calf serum, many cell lines in vitro fail to proliferate because of deprivation of growth factors that are found in neonatal serum. Because the vast majority of injected B16 cells die soon after injection, in part, because of the lack of proper growth factors, the growth of CXCR4-B16 and pLNCX2-B16 cells were examined in the presence and absence of CXCL12 under normal (10% FCS) and low (0.5% FCS) serum conditions. Under normal serum conditions, CXCR4-B16 and control cell lines showed similar rates of growth regardless of the presence of CXCL12. Under low serum conditions, however, CXCL12 enhanced the growth of CXCR4-B16 cells by ˜2-fold compared to growth without CXCL12. Moreover, the stimulated growth in the presence of CXCL12 could be blocked by the CXCR4 inhibitor, T22, but not by the control peptide, ALA. (Table 8). The presence of T22 or ALA alone did not significantly alter growth. CXCL12, T22, and ALA did not affect the growth of pLNCX2-B16 cells under either low or high serum conditions (Table 8).

TABLE 8

| | CXCR4-B16 | pLNCX2-B16 | CXCR4-B16 | pLNCX2-B16 |
|---|---|---|---|---|
| Treatment | 24 h | 24 h | 48 h | 48 h |
| PBS | 12.3 ± 2.5 | 10.7 ± 0.6 | 22.3 ± 1.2 | 21.0 ± 2.6 |
| SDF-1 | 19.3 ± 4.0 | 10.3 ± 2.1 | 35.3 ± 2.5 | 22.7 ± 0.6 |
| SDF-1 + | 12.7 ± 2.1 | 12.3 ± 0.6 | 20.7 ± 3.0 | 21.7 ± 3.2 |
| T22 | | | | |
| SDF-1 + | 16.0 ± 1.0 | 10.7 ± 2.9 | 33.3 ± 6.7 | 22.7 ± 3.2 |
| ALA4 | | | | |
| T22 | 11.0 ± 1.0 | 10.7 ± 1.2 | 22.0 ± 1.0 | 21.0 ± 1.0 |
| ALA4 | 12.0 ± 0.1 | 10.0 ± 1.0 | 22.3 ± 1.2 | 20.3 ± 3.5 |

This example demonstrated that, under low serum conditions, the expression of CXCR4 by B16 cells led to a significant enhancement of growth when CXCL12 was present in the medium.

### Example 6

This example demonstrates that CXCR4 is expressed by primary human cutaneous melanoma tumor cells as well as by melanoma cells metastatic to the lung.

To confirm the observation that melanoma cells can express CXCR4 (Robledo et al., J. Biol. Chem. 276: 45098-45105 (2001)) and to show that it can be expressed in melanoma metastatic to the lung and in primary skin tumors, fresh samples of primary and metastatic melanoma were obtained. Clear staining of a subset of cells was present in the skin and in the lung tumors with anti-CXCR4 mAb, whereas little or no signal was detected with isotype control mAb. The plasma membrane staining pattern of CXCR4 on the tumor cells is indicative of the transmembrane structure of all chemokine receptors. Two of five pulmonary metastases tested showed strong CXCR4 reactivity, while three of three primary cutaneous melanomas showed variable, but readily detectable, CXCR4 expression.

This example demonstrated that primary human cutaneous melanoma tumor cells and melanoma cells metastatic to the lung express CXCR4.

Materials

The following describes the animals, cell lines, and reagents used in Examples 7-12 described herein: Female B57BL/6 mice (8-12 weeks old) as described (Murakami et al., Cancer Res. 62:7328-7334 (2002)) were used in experiments that were approved by the NCI Animal Use and Care Committee. As previously described ((Murakami et al., Cancer Res. 62:7328-7334 (2002))), murine B16 cells were sequentially transduced with cDNA encoding either human CXCR4 (gift from Dr. E. Berger, NIAID) in the pLNCX2 retroviral vector (Clontech, Palo Alto, Calif.) or pLNCX2 alone (empty vector) and then with cDNA encoding firefly luciferase. For convenience, the CXCR4-luciferase B16 cell line is hereafter called CXCR4-B16, while the control vector-luciferase-transduced cell line is called pLNCX2-B16. CXCR4- and pLNCX2-B16 cells (Murakami et al., Cancer Res. 62:7328-7334 (2002)) were cultured in DMEM (Life Technologies, Inc., Gaithersburg, Md.) with 10% heat-inactivated fetal calf serum (cDMEM) and supplements, including puromycin and G418. Human DMEC (Emory University Department of Dermatology, Atlanta Ga.) were derived from neonatal foreskin and cultured as described (Erbe et al., J. Cell Biol. 119:215-227 (1992)). Human LMECs (Clonetics-Cambrex) were grown in EGM-2 MV (Clonetics-Cambrex). Mouse anti-human CXCR4 mAb (clone 44716.111), anti-human CXCL12 mAb (clone 77014.111), human TNF-α, VCAM-1-, E-selectin, and P-selectin-Ig chimeras were purchased from R&D Systems (Minneapolis, Minn.). Purified rat anti-mouse mAb against β₁, β₂, β₃, α₄, α_{L}, and αᵥ integrin, and FITC-conjugated mouse anti-human CD 106 (VCAM-1) were purchased from BD-PharMingen (San Diego, Calif.). The CXCR4 antagonist peptide T22 (NH₂-RRWCYRKCYKGYCYRKCR-COOH, ((Murakami et al., Cancer Res. 62:7328-7334 (2002), and Murakami et al., J. Exp. Med, 186: 1389-1393 (1997)) and an inactive control peptide (ALA, described in Murakami et al., J. Exp. Med, 186: 1389-1393 (1997)) were synthesized by Synpep (Dublin, Calif.). Chemokines were purchased from Peprotech (Rocky Hill, N.J.). A mAb (B16MAB-1) against an uncharacterized cell-surface antigen found on B16 cells was raised by immunizing hamsters with lysates of B16 cells. Hybridoma culture supernatants (Rockland Immunochemicals, Gilbertsville, Pa.) were screened for cell-surface binding activity against B16 cells by flow cytometry. One clone (B16MAB-1) showed uniform binding to B16 cells, but lacked binding to NIH 3T3 cells and murine lymph node immunocytes.

### Example 7

This example demonstrates that early treatment, but not late treatment, with T22 blocks T22 CXCR4-mediated metastasis.

It has been shown that the CXCR4 inhibitory peptide, T22, blocked pulmonary metastasis of CXCR4-B16 cells when this CXCR4 inhibitor was used daily throughout the course of the 14-day metastastis assay (Murakami et al., Cancer Res. 62:7328-7334 (2002)). To determine if short-term treatment with T22 was also effective in blocking metastasis, the in vivo assay described in Murakami et al., Cancer Res. 62:7328-7334 (2002), was modified and the drug was only given for the first two days of the 14-day waiting period following i.v. injection of CXCR4-B16 and pLNCX2-B16 cells. In one group of animals, the CXCR4 antagonist peptide T22 was administered intraperitoneally (i.p.) shortly after tumor inoculation and again 24 hours later. In another group of CXCR4-B16-injected mice, T22 was given daily for 14 days. Administration of T22 for the first two days was clearly as effective as giving T22 daily for the entire 2-week course with regard to protecting against CXCR4-mediated pulmonary metastasis.

To determine whether T22 treatment during the last 7 days of the metastasis assay could reduce tumor burden, T22 was given daily either during the entire 2-week treatment period or for just the second week (Table 9). Treatment during the second week yielded the same metastatic burden as PBS (mock) treatment; both were ineffective compared to daily T22 treatment in reducing metastasis. Thus, early treatment of CXCR-B16 cells with T22 is essential for protecting mice again lung metastasis, suggesting that CXCR4 promotes metastasis at a relatively early time point following i.v. inoculation.

TABLE 9

| | Average Relative |
|---|---|
| | Luciferase Units |
| pLNCX2/PBS | 11152.1 |
| CXCR4/PBS | 97091.1 |
| CXCR4/T22 - 2 weeks | 15294.5 |
| CXCR4/T22 - 2nd week only | 81981.2 |

This example demonstrated that T22 is effective for inhibiting metastasis when administered for only 2 days and when administered early after intravascular dissemination of tumor cells.

### Example 8

This example demonstrates the adhesion molecule profile of B16 cells and microvascular endothelial cells.

B16 cells or endothelial cells were first incubated with specific mAb (10 μg/ml) for 30-60 min at 4° C. in 0.1% BSA/PBS, washed twice, and then incubated with FITC-conjugated secondary antibody (1:100) for 30 min at 4° C. Flow cytometric analysis was performed using FloJo software (TreeStar Inc., San Carlos, Calif.).

Chemokine receptor-triggered adherence of leukocytes involves cell-surface integrins and their cognate receptors on endothelial cells. Thus, the adhesion molecule profiles of B16 cells and two types of microvascular endothelial cells were characterized. Both pLNCX2-B16 and CXCR4-B16 cells expressed the β₁ and α₄ integrin chains, but not β₂, β₇, or α_{L} (Cd11a) chains. E-selectin ligands were also not detectable after exposure of B16 cells to an E-selectin/Ig chimeric protein. Cultured LMEC expressed more VCAM-1 (a major immunoglobulin superfamily receptor for α₄β₁ integrin (VLA-4), compared to DMEC under both resting and TNF-stimulated conditions. Thus, β₁, but not β₂, integrins are expressed by B16 melanoma cells, and a major β₁ integrin ligand, VCAM-1, is readily expressed by cytokine-stimulated lung-derived endothelial cells.

This example demonstrates the adhesion molecule expression profile of B16 cells and microvascular endothelial cells.

### Example 9

This example demonstrates that CXCL12 enhances binding of CXCR4-B16 cells to soluble VCAM-1.

CXCR4-B16 or pLNCX2-B16 cells were placed in suspension (cDMEM, warmed to 37° C.) and exposed to chemokine (500 ng/ml) in the presence of VCAM-1/Ig (or P-selectin/Ig as a control). As a positive control, Mn²⁺ (2 mM) was added to the suspension to increase integrin affinity for VCAM-1. VCAM-1/IgG or P-selectin-IgG chimera (2 μg/ml) was then added to the suspension. After 45 s, cells were fixed for 10 min with 4% paraformaldehyde at RT. The cells were washed and resuspended in 0.1% BSA/PBS, and incubated with a FITC-conjugated anti-human IgG antibody at 4° C. for 30 min before flow cytometric analysis.

To determine if CXCL12 directly enhanced the binding of β₁ integrin to VCAM-1, CXCR4-B16 and pLNCX2-B16 cells were exposed to soluble VCAM-1-human IgG chimera (VCAM-1/Ig) or a P-selectin/Ig fusion protein (as a negative control) in the presence and absence of chemokines. CXCR4-B16 cells clearly demonstrated increased binding of soluble VCAM-1/IgG chimera compared to CXCL12-stimulated pLNCX2-B16 within 45 sec of the addition of CXCL12. The addition of CXCL12, but not CCL19 (an unrelated chemokine), to CXCR4-B16 cells stimulated enhanced binding of VCAM-1/Ig chimera, while the addition of CXCL12 did not affect binding of VCAM-1/Ig to pLNCX2-B16 cells. Mn²⁺ treated B16 cells uniformly increased binding of VCAM-1/Ig. Therefore, CXCL12 rapidly enhances β₁ integrin affinity for VCAM-1.

This example demonstrated that CXCL12 enhances binding of CXCR4-B16 cells to soluble VCAM-1.

### Example 10

This example demonstrates CXCL12 enhances arrest of B16-CXCR4 cells on VCAM-1/Ig-coated plates.

Cell suspension plates (35 mm, Model # 430588, Corning Inc., NY) were coated with human VCAM-1/IgG chimera (1 μg/ml) overnight at 4° C. in Tris-buffered saline (Maki et al., Immunol. 169:2346-2353 (2002)). Plates were then briefly rinsed with PBS, co-coated with 1 μg/ml of CXCL12, CCL19, or no chemokine in PBS for 2 hr at 4° C., and blocked with 1% BSA/PBS for 1 hr at 4° C. Calcein-acetomethyl (AM)-labeled (Molecular Probes, Eugene, Oreg.) CXCR4-B16 or pLNCX2-B16 cells (2.5×10⁵ cells/ml in cDMEM) were injected at 1.5 dynes/cm² into a parallel plate flow chamber (Glycotech, Gaithersburg, Md.) that had been placed over the adhesion molecule- and/or chemokine-coated suspension plate.

For dynamic flow assays using endothelial cells, LMEC at passage 5-6 were cultured to confluence in 35 mm tissue culture plates (Model # 430165, Corning Inc., NY), and treated with TNFα (10 ng/ml for 4 hr at 37° C.) prior to introduction of tumor cells. In some cases, endothelial cells were exposed to 500 ng/ml recombinant CXCL12 or CCL19 for 15 min at 20° C. as described previously (Cinamon et al., Immunol. 2:515-522 (2001)), and unbound chemokine was washed away. Where indicated, LMECs were treated with anti-CXCL12 antibody or isotype control (100 μg/ml for 30 min at 37° C.). All unbound antibody was washed off prior to infusion of B16 cells. When used, pertussis toxin (PTX, Calbiochem, San Diego, Calif.) was incubated with B16 cells at a concentration of 100 ng/ml for 2 h at 37° C. to block Gᵢ protein-coupled signaling. Where indicated, tumor cells were also treated with anti-CXCR4 mAb, hamster anti-mouse/rat β₁ integrin mAb (Ha2/5, BD-Pharmingen) and rat anti-mouse β₁ integrin (HMbl-1, BD-Pharmingen), anti-B16 mAb or isotype at 10 μg/ml for 30 min at 37° C. and washed once prior to resuspension and infusion.

For dynamic assays on recombinant molecule-coated plates as well as on LMEC, tumor cells were introduced into the chamber under a constant shear stress of 1.5 dynes/cm² without allowing cells to settle at any time during the assay. Ten min after flow was initiated, arrested cells were digitally photographed (without flow being decreased) in 4-6 random fields (each field=1.18 mm²) with excitation at 488 nm and emission at 513 nm using sufficient exposure time (˜1 s) to allow distinction between moving cells, which appeared as dim streaks, and stationary cells. Cells were then enumerated with the software program IPlab (Scanalytics, Fairfax, Va.).

Like leukocytes, tumor cells that adhere to vascular endothelial cells are subject to shear forces in vascular channels. To determine if CXCR4-CXCL12 interaction mediates the arrest of CXCR4-B16 cells on VCAM-1 under physiologic flow conditions, CXCR4-B16 or pLNCX2-B16 cells were infused over plates co-coated with VCAM-1/Ig and either CXCL12, or a control chemokine, CCL19. CXCR4-B16 cells arrested 5 times more efficiently on plates coated with VCAM-1 and CXCL12 than on plates coated with VCAM-1/Ig and CCL19 (p=0.001) (Table 11). CXCR4-B16 cells also arrested more efficiently than pLNCX2-B16 cells on VCAM-1/CXCL12-coated plates (p<0.001). Interestingly, CXCR4-B16 cells demonstrated no appreciable rolling prior to rapid binding to VCAM-1/Ig in real-time recordings. Thus, under dynamic adhesion conditions, CXCR4 expression enabled more efficient arrest of tumor cells to VCAM-1 in the presence of CXCL12.

TABLE 10

| | Mean adherent cells/field |
|---|---|
| CXCR4-B16 on VCAM + CXCL12 | 87.2 |
| CXCR4-B16 on VCAM + CCL19 | 17.2 |
| pLNCX2-B16 on VCAM + CXCL12 | 2.4 |

This example demonstrated that CXCR4 enables efficient arrest of tumor cells to VCAM-1 in the presence of CXCL12.

### Example 11

This example demonstrates that CXCL12 mediates adhesion of B16-CXCR4 cells to stimulated LMEC under physiologic shear stress.

CXCR4-B16 and pLNCX2-B16 cells in exponential growth phase were harvested by trypsinization and washed twice in PBS before injection. Cell viability was >95% as determined by trypan blue dye exclusion. CXCR4-B16 or pLNCX2-B16 cells (4×10⁵ in 200 μl PBS) were then injected into the tail veins of mice. T22 and ALA peptides in sterile PBS were administered via an i.p. route using 4 μg peptide/mouse daily for the indicated duration of treatment as described (Murakami et al., Cancer Res. 62:7328-7334 (2002)). Mice were euthanized after 14 days for gross inspection of lungs and luciferase quantification of metastasis by luciferase activity (Murakami et al., Cancer Res. 62:7328-7334 (2002)). CXCR4-B16 cells were resuspended in 0.1% BSA/PBS and treated with a hamster anti-rat β₁ integrin mAb (Ha2/5, BD-Pharmingen), a rat anti-mouse β₁ integrin mAb (9EG7, BD-Pharmingen) or as negative control, a hamster anti-B16 mAb (B16MAB-1), or rat IgG (10 μg/ml) for 30 min at 37° C. prior to injection into the parallel plate flow chamber.

A dynamic, in vitro model of B16 tumor cell adhesion to LMEC under shear stress conditions was to model the shear forces faced by tumor cells when they adhere to vascular endothelial cells during the metastatic process. CXCR4-B16 cells (vs. pLNCX2-B16 cells) arrested ˜3 times more efficiently to LMEC, even in the absence of exogenously added CXCL12 (Table 11). With exogenously added CXCL12, adhesion of CXCR4-B16 cells was ˜5-fold greater than that of pLNCX2-B 16 cells. To determine if LMEC-derived CXCL12 was essential for CXCR4-B16 arrest, LMEC were pretreated with a neutralizing anti-CXCL12 mAb, which resulted in significant inhibition of CXCR4-B16 cell arrest. An isotype control mab did not block CXCR4-B 16 binding (Table 11). As expected, pretreatment of CXCR4-B 16 cells with PTX blocked binding, indicating that a Gᵢ-coupled receptor was involved in the arrest of CXCR4-B16 cells.

TABLE 11

| | Mean adherent cells/field |
|---|---|
| CXCR4/noCXCL12 | 102.25 |
| CXCR4/+CXCL12 | 169.5 |
| CXCR4-pertussis/no CXCL12 | 42.75 |
| CXCR4-pertussis/+CXCL12 | 34.75 |
| CXCR4/anti-CXCL12 | 41.25 |
| CXCR4/mIgG | 117 |
| plncx2/no CXCL12 | 31.25 |
| plncx2/+CXCL12 | 1.25 |

To determine if β₁ integrins were critical for the arrest of B16 cells under flow conditions, CXCR4-B16 cells were pretreated with anti-β₁ integrin antibody and then injected into the flow chamber containing TNF-α-stimulated LMEC. Adherence of the tumor cells was subsequently blocked by >90% (Table 12), which was similar to the decrease in binding observed when the B16 cells were treated with anti-CXCR4 mAb. CXCR4-B16 cells treated with a surface-binding antibody (B16MAB-1) showed no significant decrease in adhesion to LMEC (Table 12), suggesting that the inhibitory effect of the anti-β₁ mAb was specific. While the epitope for B16MAB-1 binding has not yet been characterized, it is unlikely to be β₁ integrin because lymph node immunocytes, the majority of which express β₁ integrins, did not express the antigen recognized by B16MAB-1. Thus, CXCR4-mediated arrest of B16 cells on activated endothelial cells requires the participation of β₁ integrin.

TABLE 12

| | Mean adherent cells/field |
|---|---|
| CXCR4 | 133.25 |
| CXCR4/anti-CXCR4 | 7.5 |
| CXCR4/mIgG | 140.25 |
| CXCR4/anti-β₁ | 4.8 |
| CXCR4/rIgG | 92.75 |
| CXCR4/aB16 | 128.4 |
| plncx2 | 4.666667 |

Adhesion mediated by CXCR4 was dependent on shear stress, since far fewer cells adhered at 4 vs. 1.5 dynes/cm² (Table 13), and no rolling or adherence of CXCR4-B16 cells on LMEC was observed at 10 dynes/cm², consistent with other studies demonstrating optimal adherence of tumor cells and leukocytes at shear stresses less than 3 dynes/cm² (Giavanzzi et al., J. Clin. Invest. 92:3038-3044 (1993); Lawrence et al., Blood, 70:1284-1290 (1987); and Lawrence et al., J. Immunol. 151: 6338-6346 (1993)). Rolling behavior was neither observed prior to adhesion of the tumor cells at 1.5 dynes/cm², nor at higher shear stresses (up to 10 dynes/cm²). In static assays, CXCR4-B16 arrested 5 times more efficiently compared to pLNCX2-B16 on LMEC, demonstrating that, without shear stress, CXCR4 activation also results in enhanced binding of tumor cells to LMEC.

TABLE 13

Mean adherent cells/field

| | 1.5 dynes/cm² | 4 dynes/cm² |
|---|---|---|
| CXCR4 | 95.4 | 0.4 |
| pLNCX2 | 7.4 | 0.2 |

This example demonstrated that CXCL12 mediates adhesion of cells expressing CXCR4 to stimulated LMEC.

### Example 12

This example demonstrates that neutralizing anti-CXCL12 or anti-β₁ integrin mAb prevents CXCR4-mediated pulmonary metastasis.

Based on the in vitro results above, it was postulated that endothelial cell-derived CXCL12 is required for activation of CXCR4, leading to down-stream activation of β₁ integrin and subsequent firm binding of tumor cells to endothelial cell adhesion molecules such as VCAM-1. To determine if blockade of CXCL12 can suppress metastasis of B16 cells, mice were given a single dose of either neutralizing anti-CXCL12 monoclonal antibody or an isotype-matched mouse IgG isotype (i.p.) 20 min prior to i.v. injection of tumor cells. Anti-CXCL12 mAb blocked the formation of lung metastasis compared to isotype, as evidenced by the gross appearance of the lungs and by luciferase quantification (p<0.001) (Table 14). No gross metastasis in other organs (brain, liver) was observed. Therefore, neutralization of CXCL12 is effective in preventing CXCR4-mediated pulmonary metastasis.

TABLE 14

| | Luciferase Activity |
|---|---|
| pLNCX2/PBS | 674.3 |
| CXCR4/IgG | 49899.7 |
| CXCR4/anti-CXCL12 | 8327.9 |

To determine if β₁ integrin was necessary in the formation of CXCR4-mediated pulmonary metastasis in vivo, B16-CXCR4 cells were pretreated with two different blocking antibodies against β₁ integrin prior to tail vein injection. Both anti-β₁ mAbs blocked CXCR4-B16 metastasis, while rat IgG isotype control mAb did not. Pretreatment of tumor cells with the B16MAB-1 mAb also did not inhibit metastasis (Table 15), suggesting that the inhibitory effect of the β₁ integrin mAbs on metastasis was not due to non-specific effects. Thus, β₁ integrin is required for CXCR4-mediated pulmonary metastasis in vivo.

TABLE 15

| | Luciferase Activity |
|---|---|
| pLNCX2/IgG | 4544.1 |
| CXCR4/anti-B16 | 166860.4 |
| CXCR4/anti-rat B1 integrin (Ha2/5) | 21149.9 |
| CXCR4/rIgG | 204324.2 |
| CXCR4/anti-mB1 integrin (9EG7) | 37019.6 |

This example demonstrated that blocking or neutralizing CXCL12 or β₁ integrin is sufficient to inhibit metastasis of tumor cells expressing CXCR4.

### Example 13

This example demonstrates a method of inhibiting metastasis of a tumor cell, wherein the tumor cell expresses CXCR4, through use of an antagonist of α₄ integrin.

Experiments were carried out as described in Example 12, except that anti-α₄ integrin antibodies (BD-Pharmingen, San Jose, Calif.) were used in lieu of the anti-β₁ integrin antibodies. Under flow conditions, anti-α₄-antibodies were highly effective in blocking CXCL12-mediated adhesion of CXCR4-B16 cells to immobilized recombinant VCAM-1, suggesting that α₄β₁ is the primary integrin required for B16 adhesion.

This example indicates that blocking or neutralizing α₄ integrin is sufficient to inhibit metastasis of tumor cells expression CXCR4.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of inhibiting metastasis of a tumor cell in a mammal, wherein the tumor cell expresses CXC Chemokine Receptor-4 (CXCR4), which method comprises administering to the mammal in an amount sufficient to inhibit metastasis of the tumor cell a polypeptide of the formula:
Xaa₁-Xaa₂-Xaa₃Cys₄-Xaa₅-Xaa₆-Xaa₇-Cys₈-Xaa₉-Xaa₁₀-Gly₁₀-Xaa₁₂-Cys₁₃-Xaa₁₄-Xaa₁₅-Xaa₁₆-Cys₁₇-Xaa₁₈-Xaa₁₉-R (SEQ ID NO: 1)
wherein, each of Xaa₁, Xaa₂, and Xaa₁₉ is optionally a part of the polypeptide,
wherein, when Xaa₁ is a part of the polypeptide, Xaa₂ is part of the polypeptide,
wherein each of Xaa₃, Xaa₅, Xaa₉, Xaa₁₂, and Xaa₁₄ is an amino acid selected from the group consisting of Tyr, Phe, and Trp,
wherein each of Xaa₁, Xaa₂, Xaa₆, Xaa₇, Xaa₁₀, Xaa₁₅, Xaa₁₆, Xaa₁₈, and Xaa₁₉ is an amino acid selected from the group consisting of Arg and Lys,
wherein R is —OH or —NH₂,
wherein Cys₄ is optionally disulfide bonded to Cys₁₇, and
wherein Cys₈ is optionally disulfide bonded to Cys₁₃.

2. The method of claim 1, wherein the polypeptide is T22:

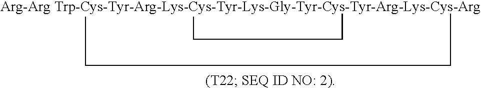

3. The method of claim 1, wherein the tumor cell can metastasize to an organ and the organ comprises cells expressing Stromal Derived Factor-1 (SDF-1).

4. The method of claim 3, wherein the organ is the skin, liver, or brain.

5. The method of claim 3, wherein the organ is the lung.

6. The method of claim 1, wherein the tumor cell is a breast cancer cell, a breast tumor cell, a lymphoma cell, a neuroblastoma cell, a lung cancer cell, an angiosarcoma cell, a pancreatic cancer cell, a leukemia cell, or a prostate cancer cell.

7. The method of claim 1, wherein the tumor cell is a melanoma cell.

8. The method of claim 7, wherein the polypeptide is administered to the mammal intraperitoneally.

9. The method of claim 1, wherein the polypeptide is administered to the mammal daily.

10. The method of claim 9, wherein the polypeptide is administered to the mammal daily for at least 2 days.

11. A method of inhibiting metastasis of a tumor cell in a mammal, wherein the tumor cell expresses a CXCR4, which method comprises administering to the mammal in an amount sufficient to inhibit metastasis of the tumor cell an antagonist of CXCR4, wherein the antagonist of CXCR4 is not an antibody that binds to CXCR4.

12. A method of inhibiting metastasis of a tumor cell in a mammal, wherein the tumor cell expresses CXCR4, which method comprises administering to the mammal in an amount sufficient to inhibit metastasis of the tumor cell an antagonist of β₁ integrin.

13. The method of claim 12, wherein the antagonist of β₁ integrin is an antibody that binds to β₁ integrin.

14. The method of claim 13, wherein the antibody that binds to β₁ integrin is a blocking antibody.

15. The method of claim 12, wherein the tumor cell is a breast cancer cell, a breast tumor cell, a lymphoma cell, a neuroblastoma cell, a lung cancer cell, an angiosarcoma cell, a pancreatic cancer cell, a leukemia cell, or a prostate cancer cell.

16. The method of claim 12, wherein the tumor cell is a melanoma cell.

17. The method of claim 12, wherein the metastasis is metastasis to the skin, liver, or brain.

18. The method of claim 12, wherein the metastasis is metastasis to the lung.

19. The method of claim 12, wherein the mammal is a human.

20. A method of inhibiting metastasis of a tumor cell in a mammal, wherein the tumor cell expresses CXCR4, which method comprises administering to the mammal in an amount sufficient to inhibit metastasis of the tumor cell an antagonist of CXCL12.

21. The method of claim 20, wherein the antagonist of CXCL12 is an antibody that binds to CXCL12.

22. The method of claim 21, wherein the antibody that binds to CXCL12 is a blocking antibody.

23. The method of claim 20, wherein the tumor cell is a breast cancer cell, a breast tumor cell, a lymphoma cell, a neuroblastoma cell, a lung cancer cell, an angiosarcoma cell, a pancreatic cancer cell, a leukemia cell, or a prostate cancer cell.

24. The method of claim 20, wherein the tumor cell is a melanoma cell.

25. The method of claim 20, wherein the metastasis is metastasis to the skin, liver, or brain.

26. The method of claim 20, wherein the metastasis is metastasis to the lung.

27. The method of claim 20, wherein the mammal is a human.

28. A method of inhibiting metastasis of a tumor cell in a mammal, wherein the tumor cell expresses CXCR4, which method comprises administering to the mammal in an amount sufficient to inhibit metastasis of the tumor cell an antagonist of α₄ integrin.

29. The method of claim 28, wherein the antagonist of α₄ integrin is an antibody that binds to α₄ integrin.

30. The method of claim 29, wherein the antibody that binds to α₄ integrin is a blocking antibody.

31. The method of claim 28, wherein the tumor cell is a breast cancer cell, a breast tumor cell, a lymphoma cell, a neuroblastoma cell, a lung cancer cell, an angiosarcoma cell, a pancreatic cancer cell, a leukemia cell, or a prostate cancer cell.

32. The method of claim 28, wherein the tumor cell is a melanoma cell.

33. The method of claim 28, wherein the metastasis is metastasis to the skin, liver, or brain.

34. The method of claim 28, wherein the organ is the lung.

35. The method of claim 28, wherein the mammal is a human.

36. A method of inhibiting growth of a tumor cell, wherein the tumor cell expresses CXCR4 and the growth is stimulated by SDF-1, which method comprises administering to the tumor cell in an amount sufficient to inhibit the growth of the tumor cell a polypeptide of the formula:
Xaa₁-Xaa₂-Xaa₃Cys₄-Xaa₅-Xaa₆-Xaa₇-Cys₈-Xaa₉-Xaa₁₀-Gly₁₁-Xaa₁₂-Cys₁₃-Xaa₁₄-Xaa₁₅-Xaa₁₆-Cys₁₇-Xaa₁₈-Xaa₁₉-R (SEQ ID NO: 1)
wherein, each of Xaa₁, Xaa₂, and Xaa₁₉ is optionally a part of the polypeptide,
wherein, when Xaa₁ is a part of the polypeptide, Xaa₂ is part of the polypeptide,
wherein each of Xaa₃, Xaa₅, Xaa₉, Xaa₁₂, and Xaa₁₄ is an amino acid selected from the group consisting of Tyr, Phe, and Trp,
wherein each of Xaa₁, Xaa₂, Xaa₆, Xaa₇, Xaa₁₀, Xaa₁₅, Xaa₁₆, Xaa₁₈, and Xaa₁₉ is an amino acid selected from the group consisting of Arg and Lys,
wherein R is —OH or —NH₂, and wherein Cys₄ is optionally disulfide bonded to Cys₁₇ and Cys₈ is optionally disulfide bonded to Cys₁₃.

37. The method of claim 36, wherein the polypeptide is T22:

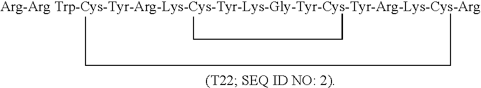

38. The method of claim 36, wherein the tumor cell is a breast cancer cell, a breast tumor cell, a lymphoma cell, a neuroblastoma cell, a lung cancer cell, an angiosarcoma cell, a pancreatic cancer, a leukemia cell, or a prostate cancer cell.

39. The method of claim 36, wherein the tumor cell is a melanoma cell.
